(19) United States
(12) Patent Application Publication
(10) Pub. No.: US 2022/0167933 A1
(43) Pub. Date: Jun. 2, 2022

(54) X-RAY IMAGING APPARATUS

(71) Applicants: Woorien Co., Ltd., Gyeonggi-do (KR); VATECH EWOO Holdings Co., Ltd., Gyeonggi-do (KR)

(72) Inventors: Byung Jun AHN, Gyeonggi-do (KR); Jiye YANG, Gyeonggi-do (KR); Jongbo YOON, Gyeonggi-do (KR); Sung-il CHOI, Gyeonggi-do (KR)

(73) Assignees: Woorien Co., Ltd., Gyeonggi-do (KR); VATECH EWOO Holdings Co., Ltd., Gyeonggi-do (KR)

(21) Appl. No.: 17/437,147

(22) PCT Filed: Mar. 9, 2020
(86) PCT No.: PCT/KR2020/003280

§ 371 Date: Sep. 8, 2021

(30) Foreign Application Priority Data
Mar. 8, 2019 (KR) 10-2019-0027181
Mar. 8, 2019 (KR) 10-2019-0027185

(51) Int. Cl. A61B 6/14; A61B 6/00; A61B 6/04; A61B 6/08

(52) U.S. Cl. CPC A61B 6/14; A61B 6/4476; A61B 6/4405; A61B 6/08; A61B 6/0407

(57) ABSTRACT

The present invention relates to an X-ray imaging apparatus which obtains an X-ray image of a subject placed between a generator and a detector by means of the rotation of the generator and the detector. The X-ray imaging apparatus comprises: the generator and the detector, which are disposed to face each other while having an object to be imaged therebetween, for irradiating and detecting an X-ray; a rotation drive unit for rotating the generator and the detector while having the object to be imaged therebetween; and a bed in which a first portion for supporting the object to be imaged is disposed between the generator and the detector. When the height of at least one from among the generator and the detector is T1, the height of the other is T2, and the height of the first portion is T3, said T1, T2, and T3 are T1>T3>T2.

### TECHNICAL FIELD

The present invention relates to an X-ray imaging apparatus and, more particularly, to an X-ray imaging apparatus acquiring an X-ray image of an object placed between a generator and a detector through rotation of the generator and the detector in such a manner as to face each other.

### BACKGROUND ART

An X-ray is an electromagnetic wave having a wavelength that is approximately one thousandth the wavelength of visible light. X-rays have a straight propagation property, a penetration property, and an attenuation property of being attenuated according to an X-ray attenuation coefficient of a material on an X-ray propagation path. An X-ray image is a radiography image that is acquired using these inherent X-ray properties. The X-ray image of an internal structure of an object is acquired on the basis of an amount of attenuated X-rays that are accumulated while X-rays pass through the object.

To this end, an X-ray imaging apparatus includes a generator emitting X-rays to the object, a detector detecting the X-rays passing through the object, and an image processing device acquiring the X-ray image of the object on the basis of a result of the detection by the detector.

In recent years, with the rapid development of semiconductor technology and information processing technology, X-ray imaging has been rapidly replaced with digital radiography (DR) that uses a digital detector. Imaging methods have also been variously developed according to purposes and applications thereof.

As an example, in an X-ray panorama image that is mostly used in the field of dentistry, a shape of an area of interest of a curved surface, that is, a shape of a set of teeth in a dental arc trace, appears in a spread-out manner on a plane. To this end, an X-ray panorama imaging apparatus obtains a two-dimensional X-ray frame image based on each section of a dental arc trace while rotating at a predetermined angle a generator and a detector, which face each other with the object in between, about a rotation shaft therebetween and moving the generator and the detector in a straight line. Then, the X-ray panorama imaging apparatus reconfigures these two-dimensional X-ray frame images using a predetermined panorama reconfiguration algorithm and thus acquires the X-ray panorama image. For reference, an overall arrangement of teeth in the dental arc trace can be easily known with the X-ray panorama image as a single image. Thus, the X-ray panorama image is known as one of the most widely used X-ray images in the field of dentistry and the like.

As another example, an X-ray computed tomography image is displayed as a 3-dimensional volume image of the object in a field of view in the form of a cylinder. To this end, a CT imaging apparatus obtains two-dimensional X-ray frame images in various direction of the object while rotating at a predetermined angle a generator and a detector, which face each other with the object in between, about a rotation shaft therebetween. Then, the CT imaging apparatus reconfigures these two-dimensional X-ray frame images using a predetermined CT algorithm, and thus acquires the X-ray CT image. For reference, the X-ray CT image may be displayed as not only a three-dimensional volume image of the object that is formed on a per-voxel basis, but also a single-surface image in a desired direction at a desired position on the basis of the three-dimensional volume image. For this reason, the X-ray CT image is widely used when exact information on an internal structure of the object is necessary, for example, when dental implant surgery is performed.

However, a general X-ray panorama imaging apparatus or CT imaging apparatus is required to rotate the generator and the detector while performing the X-ray imaging and thus needs to be configured to include large-sized complicated constituent elements.

FIG. 1 is a perspective view illustrating a general dental X-ray imaging apparatus including a column in the form of a pillar that is mounted vertically on a floor, an ascending and descending device ascending and descending along the column, a horizontal arm connected to the ascending and descending device, a gantry rotatably connected to the horizontal arm with a rotation shaft in between, and a generator and a detector that are mounted on opposite end portions, respectively, of the gantry with the rotation shaft in between. The object is placed between the generator and a sensor. A two-dimensional X-ray frame image for an X-ray panorama image or an X-ray CT image is obtained while rotation of the gantry about the rotation shaft rotates the generator and the detector around the object. The two-dimensional frame images are reconfigured in a separate image processing device not illustrated in the drawings, and thus the X-ray panorama image or the X-ray CT image is obtained.

FIG. 2 is a perspective view illustrating a general medical CT imaging apparatus including a housing with a bore hole in the form of a doughnut or an O-ring, a detector and a detector that are rotated within the housing in such a manner as to face each other with the bore hole in between, and a bed supporting the object. The object is placed within the bore hole in a state of being supported on the bed. Two-dimensional X-ray frame images in various directions of the object are obtained while the generator and the detector are rotated within the housing around the object. These two-dimensional X-ray frame images are reconfigured in a separate image processing device not illustrated in the drawings, and thus a final CT image is obtained.

In this manner, the general X-ray panorama imaging apparatus or imaging apparatus is configured to include large-sized constituent elements, and in most cases, is impossible to transport. In addition, the general X-ray panorama imaging apparatus or CT imaging apparatus is limited in the object to the human body and has the disadvantage of not being suitable for imaging an animal having a different bone structure than the human body.

### DISCLOSURE

### Technical Problem

In order to solve the above-described problems, an objective of the present invention is to provide an X-ray imaging apparatus configured to include small-sized constituent elements and suitable for X-ray imaging of not only the human body but also an animal.

### Technical Solution

In order to accomplish the above-described objective, according to an aspect of the present invention, there is provided an X-ray imaging apparatus including: a generator and a detector that are arranged in such a manner as to face each other with an object in between and that, respectively, emit and detect X-rays; a rotation drive module configured to rotate the generator and the detector with the object in between; and a bed of which a first portion supporting the object is arranged between the generator and the detector, wherein when a height of at least one of the generator and the detector is T1, a height of the other one thereof is T2, and a height of the first portion is T3, a relationship among T1, T2, and T3 satisfies the expression T1>T3>T2.

### Advantageous Effects

The X-ray imaging apparatus according to the present invention has the advantage of being configured to include small-sized constituent elements and being capable of X-ray imaging of an object. Particularly, the X-ray imaging apparatus according to the present invention has the advantage of being suitable for X-ray imaging of not only a human, but also an animal, particularly, a dog having a different bone structure than a human.

### DESCRIPTION OF DRAWINGS

FIG. 1 is a view illustrating a general dental X-ray imaging apparatus;

FIG. 2 is a view illustrating a general medical CT imaging apparatus;

FIGS. 3 to 5 are views illustrating an X-ray imaging apparatus according to the present invention, when viewed from different directions; and

FIGS. 6 to 8 are views each illustrating internal structures of some constituent elements of the X-ray imaging apparatus according to the present invention.

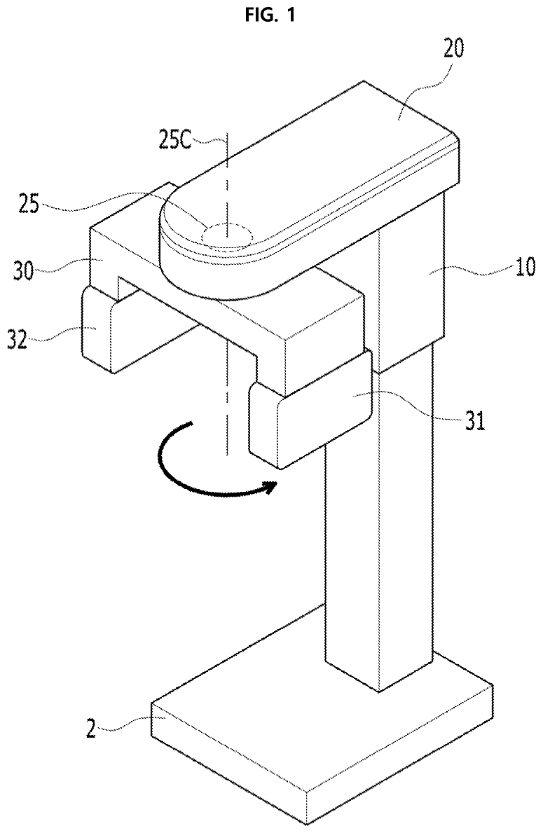

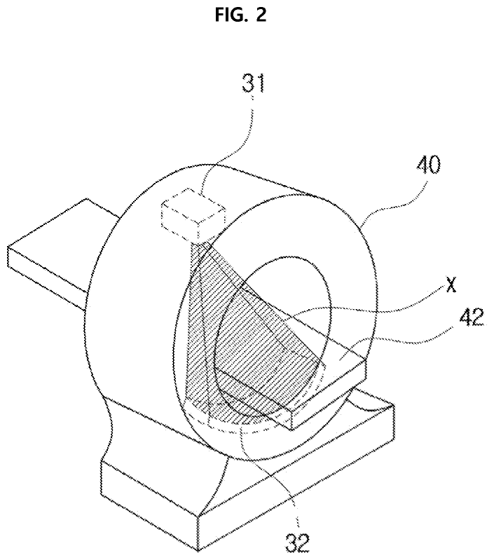

### MODE FOR INVENTION

A desired embodiment of the present invention will be described below with reference to the drawings.

For reference, the desired embodiment of the present invention will be described as a physical embodiment of the technical idea of the present invention for illustrative purpose, and the present invention is not limited to the desired embodiment. Likewise, for convenience in description, several illustrative examples and assumptions may be provided below, but the present invention is not limited to these. That is, various modifications and applications may be made to embodiments of the present invention within the scope of the technical idea of the present invention and would be easily understood from the following description. In addition, modification and applications may also be made to the drawings that serve as references for understanding the technical idea of the present invention. Relative sizes of constituent elements illustrated in the drawings may be different than is actually the case.

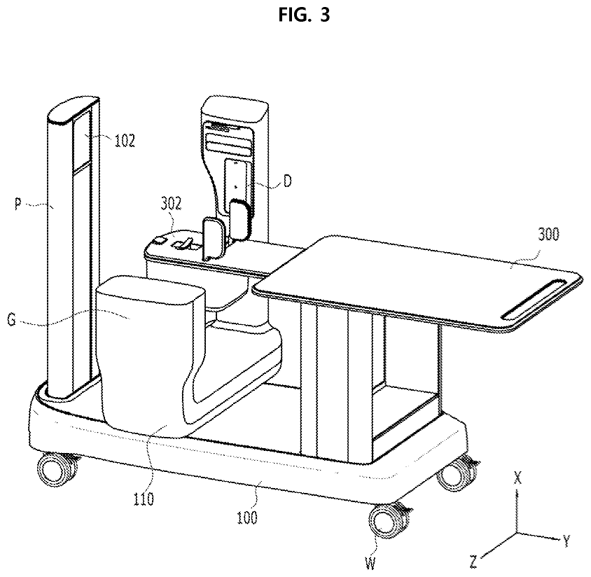

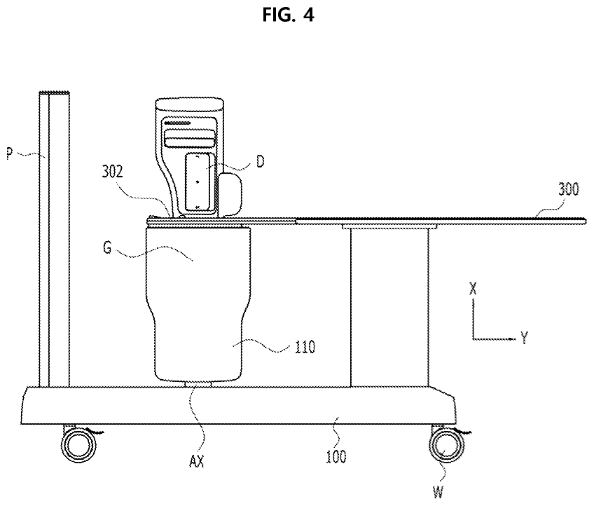

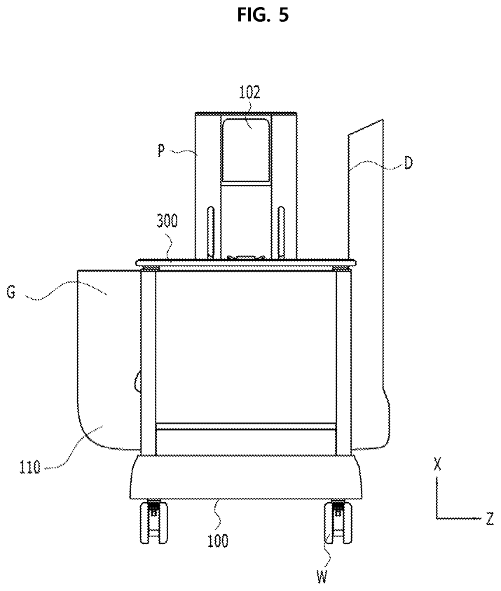

FIGS. 3 to 5 are views illustrating an X-ray imaging apparatus according to the present invention, when viewed from different directions, respectively. FIG. 3 is a perspective view illustrating the X-ray imaging apparatus. FIG. 4 is a front view illustrating the X-ray imaging apparatus in the X-Y plane. FIG. 5 is a side view illustrating the X-ray imaging apparatus in the Y-Z plane.

The X-ray imaging apparatus according to the present invention includes: a generator and a detector that are arranged in such a manner as to face each other with an object in between and, respectively, emit and detect X-rays; a rotation drive module rotating the generator and the detector with the object in between; and a bed of which a first portion supporting the object is arranged between the generator and the detector, wherein when a height of at least one of the generator and the detector is T1, a height of the other one thereof is T2, and a height of the first portion is T3, a relationship among T1, T2, and T3 satisfies the expression T1>T3>T2.

More specifically, as illustrated in the drawings, an X-ray imaging apparatus according to the present invention includes: a base frame 100 placed on a floor; a support AX protruding upward from the base frame 100; a generator G and a detector D that are arranged in such a manner as to face each other with the support AX in base and that, respectively, emit and detect X-rays; a rotation drive module 200 rotating the generator G and the detector D about the support AX in such a manner as to face each other; a bed 300 of which a first portion 302 supporting an object is arranged between the generator G and the detector D, and a controller, although not illustrated in the drawings, controlling operation of each of these constituent elements.

The base frame 100 has a supportive structure supporting an entire substantial weight of the X-ray imaging apparatus according to the present invention. The base frame 100 provides a plane on which the other constituent elements described below are mounted. It is desirable that at least one wheel W may be mounted on the base frame 100. The wheel W, supported on the floor, may support the base frame 100. A user may easily move or transport the X-ray imaging apparatus according to the present invention using the wheel W.

The support AX protrudes upward from the base frame 100. The support AX serves as a type of pillar. On the assumption that the base frame 100 is in the horizontal plane, the support AX may be arranged along the X-axis direction perpendicular to the base frame 100. However, the support AX is not limited to this arrangement. As long as the support AX does not get in the way of rotating the detector D, it is also possible that the support AX is arranged in a manner that is inclined at or below a predetermined angle with respect to the X-axis.

The generator G and the detector D are arranged towards opposite sides, respectively, of the support AX and are rotated by the rotation drive module in such a manner as to face each other. To this end, the X-ray imaging apparatus according to the present invention may include a gantry 110 that is a predetermined mechanical structure connected to the support AX. The generator G and the detector D are arranged on opposite end portions, respectively, of the gantry 110 and are rotated about the support AX therebetween in such a manner as to face each other.

A detailed structure of the rotation drive module will be described separately in more detail below with reference to the related drawings.

The first portion 302 of the bed 300 supports the object. For reference, throughout the present specification, the object means a portion on which the X-ray imaging apparatus according to the present invention actually performs X-ray imaging. The object may be a portion of an object to inspection. As an example, when the object to inspection is an animal, particularly, a dog, the object may be a dog's head. Whenever necessary, it is also possible that an entire object to inspection is placed on the bed 300. In this case, a portion of the bed 300 which supports the dog's head that is the object is referred to as the first portion 302. In this case, at least the first portion 302 of the bed 300 may be formed of a radioactive-ray transmitting material and may be a carbon plate formed of a carbon material. The bed 300 is mounted fixedly on the base frame 100. It is also possible that the bed 300 is provided separately from the base frame 100.

In the X-ray imaging apparatus according to the present invention, when the height of one of the generator G and the detector D is T1, the height of the other one thereof is T2, and the height of the first portion 302 of the bed 300 is T3, the relationship among T1, T2, and T3 satisfies the expression T1>T3>T2.

In other words, in the X-ray imaging apparatus according to the present invention, the respective heights of the generator G and the detector D are different from each other. The first portion 302 of the bed 300 is arranged in the height direction between the generator G and the detector D. The detector D is illustrated as being higher than the generator G. However, the opposite may also be true. It is also possible that the generator G is higher than the detector D.

Therefore, with rotation of the gantry 110, the generator G or the detector D, whichever has a lower height, passes below the bed 300, and the generator G or the detector D, whichever has a greater height, is rotated around the bed 300 from one side to the other side in a manner that is far enough away from the bed 300 to avoid a collision therewith.

During this rotation, at least some of the X-rays emitted by the generator G pass through the first portion 302 of the bed 300 and the object, which is placed on the first portion 302, in a direction inclined upward or downward from the horizontal. Then, the detector D detects the X-rays that pass through the object in the direction inclined upward or downward from the horizontal.

As a result, the X-ray imaging apparatus according to the present invention is suitable for X-ray imaging of not only the human body, but also an animal having a different bone structure than the human body. More specifically, not only a head of an animal, particularly a dog, but also a dental arc thereof has a different structure than the human body. Anterior and posterior portions of an upper jaw bone of the dog have different heights. A palate of the dog has a lower height than the human palate. A portion constituting the palate, of the upper jaw bone of the dog is not quite so high, compared with heights of roots of upper jaw teeth. For this reason, as is the case with X-ray imaging of the head of the human body, when X-ray imaging is performed with substantially horizontal X-rays, particularly when an X-ray panorama image is captured with substantially horizontal X-rays, X-rays passing through root portions of teeth pass through a thick bone portion of the upper jaw bone, such as the portion constituting the palate, thereby increasing an X-ray attenuation ratio due to the thick bone portion. Thus, a so-called artifact appears. In the artifact, tooth-root portions of posterior teeth of an upper jaw and tooth portions of anterior teeth have a white color.

For this reason, the X-ray imaging apparatus according to the present invention emits X-rays in such a manner as to pass through the object in the direction inclined downward or upward from the horizontal and thus is suitable for the X-ray imaging of an animal, as well as the human body. For reference, from the perspective of a mechanical design, it is advantageous that the height of a relatively heavy generator G is T2, that is, that the generator G has a smaller height than the direction D. However, the generator G is not limited to this height. As described above, it is also possible that the height of the direction D is T2, that is, that the direction D has a smaller height than the generator 1G.

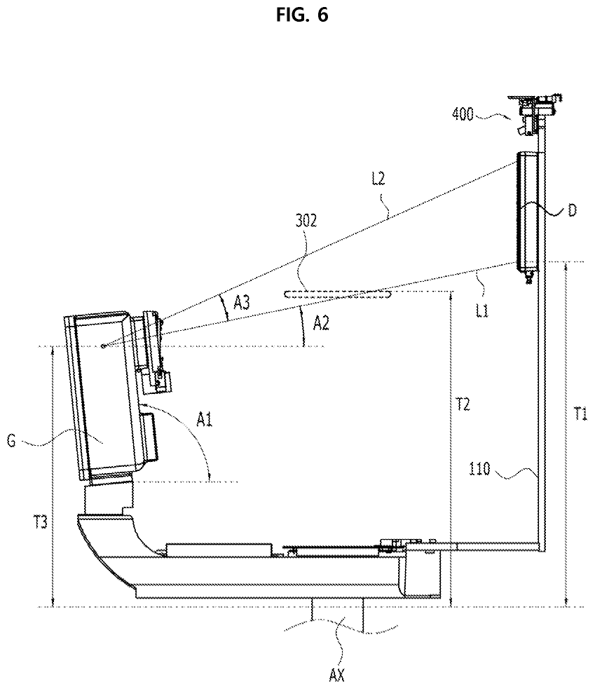

FIG. 6 is a view illustrating some constituent elements of the X-ray imaging apparatus according to the present invention. FIG. 6 illustrates a relative position of the first portion 302 with respect to the generator G, the detector D, and the bed 300. For reference, FIG. 6 illustrates the gantry 110 from which a casing is removed and the generator G and the detector D that are mounted on the gantry 110. For convenience, the first portion 302 of the bed 300 is indicated by a dotted line.

As illustrated, in the X-ray imaging apparatus according to the present invention, as an example, the respective heights of the direction D, the generator G, and the first portion 302 of the bed 300 are T1, T2, and T3, respectively, the relation among the respective heights thereof satisfy the expression T1>T3>T2. In this case, the generator G may be arranged obliquely toward the detector D in a manner that is inclined at a first range A1 of angles from 91 to 100 degrees, desirably, at a range of angles from 93 to 97 degrees. Line L1 indicating the lowest X-ray in the X-ray beam emitted by the generator G may be inclined upward at a second range A2 of angles from 5 to 15 degrees, desirably, at a range of angles from 8 to 13 degrees with respect to the horizontal. Then, a range of angles of the X-ray ray beam emitted by the generator G, that is, a range A3 of angles between Line L1 indicating a lowermost X-ray in the X-ray beam emitted by the generator G and Line L2 indicating an uppermost X-ray in the X-ray beam may be from 10 to 30 degrees, desirably, from 10 to 15 degrees.

For reference, the height of the detector D is defined as a height from the horizontal to an uppermost end of the direction D, the height of the generator G is defined as a height from the horizontal to a focal spot, and the first portion 302 of the bed 300 is defined as a height from the horizontal to an upper surface of the first portion 302. The numerical values of the heights are determined on the assumption that the object is an ordinary person. Therefore, it is possible that the heights and the numerical values thereof are changed without any restriction whenever necessary.

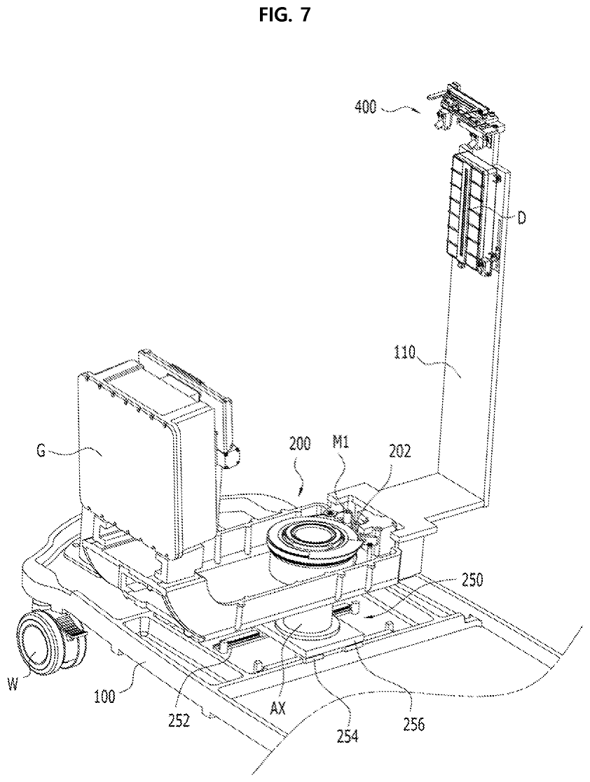

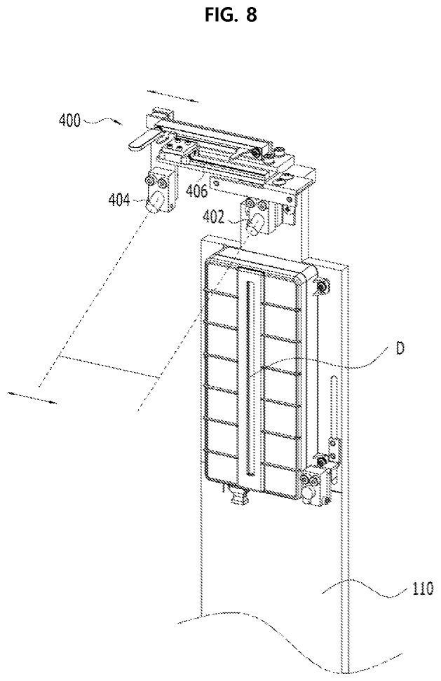

FIG. 7 is a view of other constituent elements of the X-ray imaging apparatus according to the present invention. FIG. 7 illustrates the detailed structure of the rotation drive module 200. For reference, FIG. 8 also illustrates only necessary constituent elements of the X-ray imaging apparatus according to the present invention.

As illustrated, the rotation drive module 200 includes the gantry 110 that is a predetermined mechanical structure connected to the support AX. The generator G and the detector D are arranged on opposite end portions, respectively, of the gantry 110 in the middle of which the support AX is positioned. As the gantry 110 is rotated, the generator G and the detector D are rotated about the support AX.

At this time, an end portion of the support AX may pass through the gantry 110 in a state where the support AX is stationary. The rotation drive module 200 includes a motor fixed to the gantry 110, and a power transfer member 202 that connects a rotation shaft M1 of the motor and the support AX. The rotation shaft M1 of the motor may be rotated along with the support AX, and the power transfer member 202 may be a timing belt. Therefore, when the rotation shaft M1 of the motor is rotated, the gantry 110 is rotated about the support AX with a rotational force thereof. For reference, the power transfer member 202 is not limited to the timing belt. Any power transfer member, such as a gear, that is capable of transferring the rotational force of the rotation shaft M1 of the motor and thus rotating the gantry 110 may be employed without any particular restriction.

With the rotation of the gantry 110, the X-ray imaging apparatus according to the present invention is capable of performing CT imaging on the object. For reference, the CT imaging is performed while the generator G the detector D are rotated about a single axis in such a manner as to face each other with the object in between. As a result, frame images in various direction of the object are obtained. These frame images are reconfigured with a predetermined CT algorithm. As a result of the reconfiguration, a CT image is obtained. The X-ray imaging apparatus according to the present invention also rotates the gantry 110 about the support AX using the rotation drive module 200 and thus rotates the generator G and the detector D in such a manner as to face each other with the object in between. Thus, X-ray frame images in various directions of the object may be obtained with generator G and the detector D. When these X-ray frame images are reconfigured with a predetermined CT algorithm, a CT image of the object may be obtained. The direction D for obtaining the X-ray frame images may be a detector for CT imaging in the form of a square or a rectangle.

It is desirable that the X-ray imaging apparatus according to the present invention further includes a liner drive module 250 moving the gantry 110 in a straight line in at least one direction with respect to the base frame 100 for panorama imaging of the object. For reference, the panorama image is obtained as a result of reconfiguring frame images, which of each is based on each section of a dental arc trace, using a predetermined panorama algorithm. For this reason, magnification ratios of the section-based frame images are similar to each other. To this end, while performing the panorama imaging, the detector D needs to be moved in a shape similar to that of the dental arc trace along an edge of the dental arc trace. However, because the dental arc trace is in the form of an ellipse, the gantry 110 needs to be both rotated and moved in a straight line while performing the panorama imaging.

The X-ray imaging apparatus according to the present invention is capable of moving the support AX in a straight line in at least one direction with respect to the base frame 100 through the liner drive module 250.

To this end, as an example, the liner drive module 250 may include a linear guide 252, a carriage plate 254, and a linear motor. The linear guide 252 is mounted on the base frame 100. The support AX is connected to the carriage plate 254, and the carriage plate 254 is movable along the linear guide 252. The linear motor moves the carriage plate 254 along the linear guide 252. In this case, it is desirable that linear guides 252 constituting a pair are in parallel to each other and that a pair of carriage plates 254 is provided along each of the linear guides 252. A moving plate 256 in the form of a plate is interposed between the support AX and the carriage plate 254. The support AX is fixed on an upper surface of the moving plate 256, and the carriage plate 254 is combined with a lower surface of the moving plate 256. Thus, an eccentric load due to the rotation and the straight-line movement of the gantry 110 while performing the X-ray imaging may be suitably distributed to a plurality of carriage plates 254.

A direction of straight-line movement of the support AX may be a first direction that is a forward-backward direction of the dental arc or may be a second direction perpendicular to the forward-backward direction of the dental arc. In a case where the linear guide 252 is arranged along the first direction that is the forward-backward direction of the dental arc, the direction of the straight-line movement of the support AX is the first direction that is the forward-backward direction of the dental arc. In a case where the linear guide 252, as illustrated in the drawings, is the second direction perpendicular to the forward-backward direction of the dental arc, the direction of the straight-line movement of the support AX is the second direction perpendicular to the forward-backward direction of the dental arc.

Therefore, the X-ray imaging apparatus according to the present invention may perform the panorama imaging while moving the detector D in such a manner as to form a shape similar to that of the dental arc trace along the edge of the dental arc trace in conjunction with the rotation and the straight-line movement of the gantry 110. To this end, the detector D may be a slit-shaped panorama-dedicated detector whose length is larger than a width.

A dental arc trace of an animal, particularly a dog varies in size and shape from species to species. For this reason, the rotation and the straight-line movement of the gantry 110 for the panorama imaging need to be suitably controlled. Particularly, the straight-line movement needs to be changeable according to a size of the dental arc.

To this end, the X-ray imaging apparatus according to the present invention provides a dental-arc measurement means capable of measuring a length of a dental arc of the object in advance before performing the X-ray imaging. The controller adjusts the rotation of the gantry 110 and a distance by which the gantry 110 is moved in a straight line, in one of several imaging modes that are predetermined according to a length of the dental arc measured by the dental-arc measurement means.

FIG. 8 is a view illustrating a dental-arc measurement means 400 of the X-ray imaging apparatus according to the present invention.

As illustrated, the dental-arc measurement means 400 includes a first beam 402 and a second beam 404. The first beam emits visible light to a first reference position on the object from one side of the object. The second beam is capable of adjusting a gap between to the first and second beams 402 and 404 along the forward-backward direction of the dental arc and emits visible light to a second reference position on the object from one side of the object.

In this case, it is desirable that the first and second beams 402 and 404 may be mounted on the gantry 110 and that, on the assumption that the generator G is positioned at a smaller height than the detector D, the first beam 402 may be fixedly mounted on an upper end portion of the detector D for user convenience. The second beam 404 is provided in a manner that is movable along a guide 406 extending horizontally along the forward-backward direction of the dental arc from the first beam 402. A sensor measuring the gap between the first and second beams 402 and 404 is built into the guide 406. In a case where the object is an ordinary person, the first reference position may be one of an eye, a molar tooth, and an infraorbital foramen, and the second reference position may be an anterior tooth.

Therefore, in order to determine the gap between the first and second beams 402 and 404, a user safely places the object on the bed 300 in such a manner that the first reference position is aligned to the first beam 402 and thus adjusts a position of the second beam 404 in such a manner that the second beam 404 emits visible light to the second reference position. In this manner, when the gap between the first and second beams 402 and 404 is determined, the controller rotates the gantry 110 and moves the gantry 110 in a straight line in a sequence of operations according to an imaging mode that is preset on a per-dental arc basis and thus performs the panorama imaging of the object. At this time, when a length in the forward-backward direction of the dental arc is increased, a distance by which the gantry 110 is moved in a straight line is increased while performing the panorama imaging. Furthermore, when the length in the forward-backward direction of the dental arc is decreased, the distance by which the gantry 110 is moved in a straight line is decreased while performing the panorama imaging. That is, the length in the forward-backward direction of the dental arc and the distance by which the gantry 110 is moved in a straight line while performing the panorama imaging are in proportion to each other.

Two-dimensional X-ray frame images, each of which is based on each section of the trace of the dental arc of the object, are obtained in this manner. These two-dimensional X-ray frame images are reconfigured using a predetermined panorama reconfiguration algorithm, and thus the X-ray panorama image is provided.

1-11. (canceled)

12. An X-ray imaging apparatus comprising:
a generator for irradiating X-rays toward a object;
a detector for detecting X-rays passed through the object;
a rotation drive module for rotating the generator and the detector based on the object; and
a support part for positioning the object between the generator and the detector,
wherein the height of one of the generator and the detector is T1, the height of the other is T2, and the height of the support part is T3, the relationship between T1, T2, and T3 is T1>T3>T2, and
the height of the rotation center of the rotation drive module is lower than the T2.

13. The X-ray imaging apparatus of claim 12, wherein the lower one of the generator and the detector passes below the support part by the rotation.

14. The X-ray imaging apparatus of claim 12, wherein at least some of X-rays emitted from the generator pass through the support part.

15. The X-ray imaging apparatus according to claim 12, further comprising;
a base frame placed on the floor, wherein the rotation drive module is rotatably mounted on the base frame.

16. The X-ray imaging apparatus of claim 15, further comprising;
a linear drive module provided on the base frame, wherein the linear drive module linearly moves the rotation drive module.

17. The X-ray imaging apparatus of claim 16, wherein the object is a head including a dental arc.

18. The X-ray imaging apparatus of claim 17, wherein the linear drive module moves the rotation drive module in a straight line along a first direction that is a forward-backward direction of the dental arc or a second direction perpendicular to the forward-backward direction of the dental arc.

19. The X-ray imaging apparatus of claim 18, further comprising:
a dental-arc measurement module configured to measure a length in the forward-backward direction of the dental arc,
wherein the liner drive module moves the rotation drive module in a straight line along the length in the forward-backward direction of the dental arc while performing X-ray imaging.

20. The X-ray imaging apparatus of claim 19, wherein the dental-arc measurement module comprises:
a first beam configured to emit visible light to a first reference position on the object; and
a second beam configured to being capable of adjusting a gap between the first and second beams along the forward-backward direction of the dental arc and to emit visible light to a second reference position on the object;
wherein the liner drive module adjusts the distance by which the rotation drive module is moved in a straight line, according to the gap between the first and second beams.

21. The X-ray imaging apparatus of claim 20, wherein the first and second beams are mounted on the rotation drive module.

22. The X-ray imaging apparatus of claim 20, wherein the first reference position is one of an eye, a molar tooth, and an infraorbital foramen, and the second reference position is an anterior tooth.
